(19) United States
(12) Patent Application Publication
(10) Pub. No.: US 2018/0003677 A1
(43) Pub. Date: Jan. 4, 2018

(54) PIEZOELECTRIC PACKAGE-INTEGRATED CHEMICAL SPECIES-SENSITIVE RESONANT DEVICES

(71) Applicant: Intel Corporation, Santa Clara, CA (US)

(72) Inventors: Sasha N. OSTER, Chandler, AZ (US); Feras EID, Chandler, AZ (US); Georgios C. DOGIAMIS, Chandler, AZ (US); Thomas L. SOUNART, Chandler, AZ (US); Adel A. ELSHERBINI, Chandler, AZ (US); Johanna M. SWAN, Scottsdale, AZ (US); Shawna M. LIFF, Scottsdale, AZ (US)

(73) Assignee: Intel Corporation, Santa Clara, CA (US)

(21) Appl. No.: 15/199,901

(22) Filed: Jun. 30, 2016

(51) Int. Cl. G01N 29/02; B81B 3/00; G01N 33/00; G01N 33/543; G01N 33/22; G01N 29/036

(52) U.S. Cl. CPC G01N 29/022; G01N 33/227; G01N 29/036; G01N 33/0047; G01N 33/54373; B81B 3/0021; B81B 2201/0214; G01N 2291/0256; G01N 2291/0423

(57) ABSTRACT

Embodiments of the invention include a chemical species-sensitive device that includes an input transducer to receive input signals, a base structure that is coupled to the input transducer and positioned in proximity to a cavity of an organic substrate, a chemically sensitive functionalization material attached to the base structure, and an output transducer to generate output signals. For a chemical sensing functionality, a desired chemical species attaches to the chemically sensitive functionalization material which causes a change in mass of the base structure and this change in mass causes a change in a mechanical resonant frequency of the chemical species-sensitive device.

### FIELD OF THE INVENTION

Embodiments of the present invention relate generally to package integrated acoustic transducer devices. In particular, embodiments of the present invention relate to piezoelectric package integrated chemical species-sensitive resonant devices.

### BACKGROUND OF THE INVENTION

As computing technology becomes more ubiquitous, there is a growing demand for low-cost, pervasive sensing. In particular, chemical sensing is an area of strong interest. Current chemical sensors are often bulky and are always external components that must be attached to the system on chip (SOC) package or system board and routed to the SOC.

### BRIEF DESCRIPTION OF THE DRAWINGS

FIG. 1 illustrates a view of a microelectronic device 100 having a package-integrated piezoelectric transducer device, according to an embodiment.

FIG. 2 illustrates a top view of a package substrate having a package-integrated piezoelectric chemical species-sensitive resonant device, according to an embodiment.

FIG. 3 illustrates a side view of a package substrate having a package-integrated piezoelectric chemical species-sensitive resonant device, according to an embodiment.

FIG. 4 illustrates a side view of a package substrate having a package-integrated piezoelectric chemical species-sensitive resonant device, according to an embodiment.

FIG. 5 illustrates a top view of a package substrate having a package-integrated piezoelectric chemical species-sensitive resonant device, according to an embodiment.

FIG. 6 illustrates a top view of a package substrate having a package-integrated piezoelectric chemical species-sensitive resonant device, according to an embodiment.

FIG. 7 illustrates a side view (cross-sectional view AA of FIG. 6) of a package substrate having a package-integrated piezoelectric chemical species-sensitive resonant device, in accordance with one embodiment.

FIG. 8 illustrates a side view of a package substrate having a package-integrated piezoelectric chemical species-sensitive resonant device, according to an embodiment.

FIG. 9 illustrates a top view of a package substrate having a package-integrated piezoelectric chemical species-sensitive resonant device, according to an embodiment.

FIG. 10 illustrates a computing device 1500 in accordance with one embodiment.

### DETAILED DESCRIPTION OF THE INVENTION

Described herein are piezoelectric package integrated chemical species-sensitive resonant devices. In the following description, various aspects of the illustrative implementations will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative implementations. However, it will be apparent to one skilled in the art that the present invention may be practiced without the specific details. In other instances, well-known features are omitted or simplified in order to not obscure the illustrative implementations.

Various operations will be described as multiple discrete operations, in turn, in a manner that is most helpful in understanding the present invention. However, the order of description should not be construed to imply that these operations are necessarily order dependent. In particular, these operations need not be performed in the order of presentation.

The present design provides thin, low cost, small form factor chemical species-sensitive resonant devices that are manufactured as part of an organic package substrate traditionally used to route signals between the CPU or other die and the board. The chemical species-sensitive resonant devices are fabricated utilizing substrate manufacturing technology. These chemical species-sensitive resonant devices include suspended base structures (e.g., membranes) that are free to move and are mechanically coupled to a piezoelectric material.

Conventional continuous chemical sensing techniques typically use a PID (photoionization detector), a MOS (Metal Oxide Semiconductor) sensor, or quartz crystals. All sensor devices employing these schemes are packaged separately or fabricated on more expensive Silicon substrates. The advantages of using package-integrated resonant sensors include significantly smaller form factors and direct integration with the package, with no need for assembly of a discrete component. Also, fabrication in low-cost package substrates using panel-level processing results in a significant cost savings over existing chemical sensors.

In addition, when compared to PID sensors, the advantages of using package-integrated, resonant sensors include much lower concentration detection limits (e.g., 1-10 parts per billion) and very high selectivity (e.g., selectivity for oxygen gas, nitrogen gas, desired chemical species, etc.), which is known to be a concern with PID sensors that typically suffer from poor selectivity. For a controlled indoor environment, a sensor can be designed based on knowing which chemical species will be present in this environment.

When compared to MOS sensors, additional advantages of using package-integrated, resonant sensors have better sensitivity and the capability of obtaining a linear response in the concentrations of interest. In comparison to electromagnetic transduction, the integrated piezoelectric design allows more compact form factors by eliminating the need for assembling components such as permanent magnets to the substrate.

The present design can be manufactured as part of the substrate fabrication process with no need for purchasing and assembling discrete components. It therefore enables high volume manufacturability (and thus lower costs) of systems that need sensing of chemical species. Package substrate technology using organic panel-level (e.g., ˜0.5 m×0.5 m sized panels) high volume manufacturing (HVM) processes has significant cost advantages compared to silicon-based MEMS processes since it allows the batch fabrication of more devices using less expensive materials. However, the deposition of high quality piezoelectric thin films has been traditionally limited to inorganic substrates such as silicon and other ceramics due to their ability to withstand the high temperatures required for crystallizing those films. The present design is enabled by a new process to allow the deposition and crystallization of high quality piezoelectric thin films without degrading the organic substrate.

In one example, the present design includes package-integrated structures to act as chemical species-sensitive resonant devices. Those structures are manufactured as part of the package layers and are made free to vibrate or move by removing the dielectric material around them. The structures include piezoelectric stacks that are deposited and patterned layer-by-layer into the package. The present design includes creating chemical species-sensitive resonant devices in the package on the principle of suspended and vibrating structures. Etching of the dielectric material in the package occurs to create cavities. Piezoelectric material deposition (e.g., 0.5 to 1 um deposition thickness) and crystallization also occur in the package substrate during the package fabrication process. An annealing operation at a substrate temperature range (e.g., up to 260° C.) that is lower than typically used for piezoelectric material annealing allows crystallization of the piezoelectric material (e.g., lead zirconate titanate (PZT), potassium sodium niobate (KNN), aluminum nitride (AlN), zinc oxide (ZnO), etc) to occur during the package fabrication process without imparting thermal degradation or damage to the substrate layers. In one example, laser pulsed annealing occurs locally with respect to the piezoelectric material without damaging other layers of the package substrate (e.g., organic substrate) including organic layers.

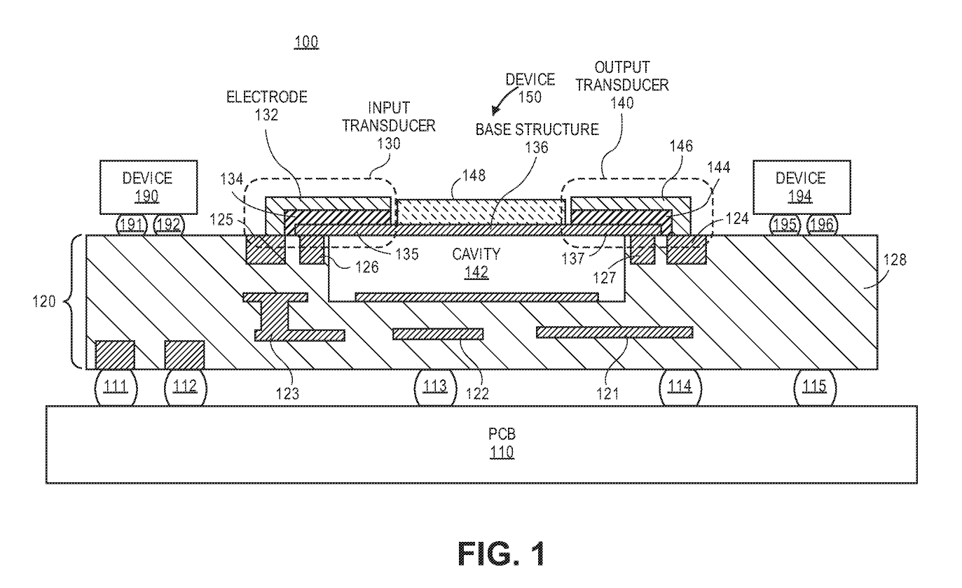

Referring now to FIG. 1, a view of a microelectronic device 100 having package-integrated piezoelectric devices is shown, according to an embodiment. In one example, the microelectronic device 100 includes multiple devices 190 and 194 (e.g., die, chip, CPU, silicon die or chip, radio transceiver, etc.) that are coupled or attached to a package substrate 120 with solder balls 191-192, 195-196. The package substrate 120 is coupled or attached to the printed circuit board (PCB) 110 using, for example, solder balls 111 through 115.

The package substrate 120 (e.g., organic substrate) includes organic dielectric layers 128 and conductive layers 121-127. Organic materials may include any type of organic material such as flame retardant 4 (FR4), resin-filled polymers, prepreg (e.g., pre impregnated, fiber weave impregnated with a resin bonding agent), polymers, silica-filled polymers, etc. The package substrate 120 can be formed during package substrate processing (e.g., at panel level). The panels formed can be large (e.g., having in-plane (x, y) dimensions of approximately 0.5 meter by 0.5 meter, or greater than 0.5 meter, etc.) for lower cost. A cavity 142 is formed within the packaging substrate 120 by removing one or more layers (e.g., organic layers, dielectric layers, etc.) from the packaging substrate 120. In one example, a piezoelectric input transducer device 130 is formed with conductive structures 132 and 136 (e.g., cantilevers, beams, traces) and piezoelectric material 134. The three structures 132, 134, and 136 form a stack. The conductive structure 132 can act as a second electrode and a region 135 of the conductive base structure 136 can act as a first electrode of the piezoelectric vibrating input device. A piezoelectric output transducer device 140 is formed with conductive structures 146 and 136 (e.g., cantilevers, beams, traces) and piezoelectric material 144. The three structures 146, 144, and 136 form a stack. The conductive structure 146 can act as a second electrode and a region 137 of the conductive base structure 136 can act as a first electrode of the piezoelectric vibrating output device. The cavity 142 can be air filled.

The base structure 136 (e.g., membrane 136) is free to vibrate in a vertical direction (e.g., along a z-axis). It is anchored on the cavity edges by package vias 126 and 127 which serve as both mechanical anchors as well as electrical connections to the rest of the package. For chemical sensing, the base structure is functionalized with a chemically selective receptor layer 148 (e.g., functionalization material). This layer could be any coating that would preferentially adsorb or absorb a specific compound (or class of compounds) of interest at a higher percentage than other components in the ambient.

In a sensing mode for a chemical species-sensitive resonant device 150, in one example of operation, an input transducer 130 is excited by applying a time varying (e.g., AC) voltage to the piezoelectric material 134 which causes it to deform. This causes vibrations of the base structure 136 that are transmitted to an output transducer 140, which generates an electrical output signal. By measuring the amplitude of this output signal at different input frequencies, or alternatively by using a feedback loop (e.g., phase locked loop), a mechanical resonant frequency of the device 150 at which the output electrical signal amplitude is maximized can be determined. When the desired chemical species attach to the material 148, which is disposed on the base structure 136, the mass of the base structure changes, which in turn changes the mechanical resonant frequency. This shift in frequency is detected in the electrical signals and is used to correlate the amount of a specific chemical species present in the environment.

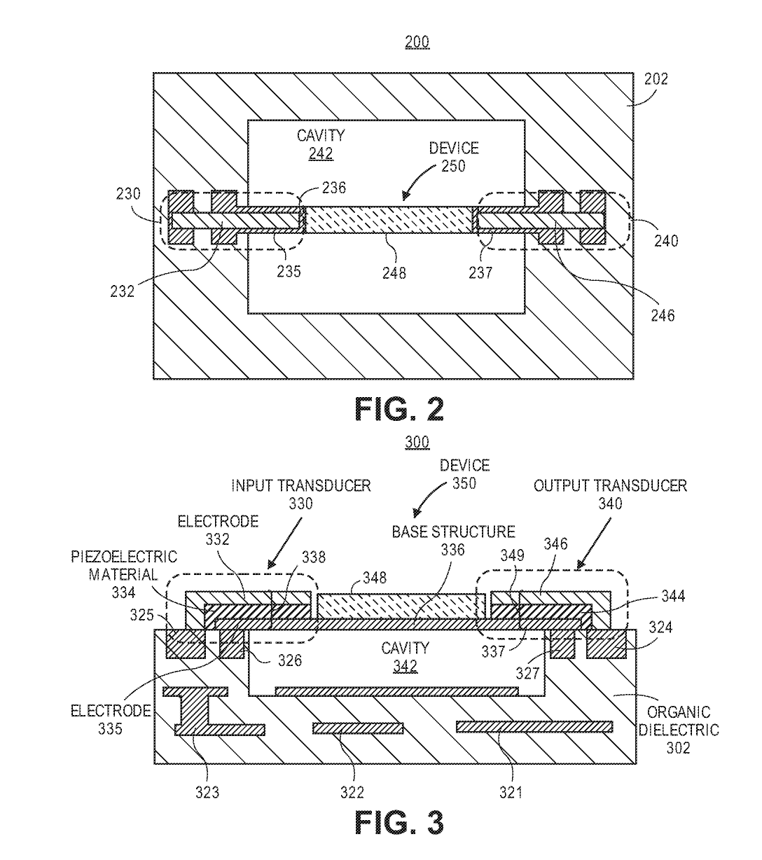

FIG. 2 illustrates a top view of a package substrate having a package-integrated piezoelectric chemical species-sensitive resonant device, according to an embodiment. In one example, the package substrate 200 may be coupled or attached to multiple devices (e.g., die, chip, CPU, silicon die or chip, RF transceiver, etc.) and may be also coupled or attached to a printed circuit board (e.g., PCB 110). The package substrate 200 (e.g., organic substrate) includes organic dielectric layers 202 and conductive layers 232, 236, and 246. The package substrate 200 can be formed during package substrate processing (e.g., at panel level). A cavity 242 is formed within the packaging substrate 200 by removing one or more layers (e.g., organic layers, dielectric layers, etc.) from the packaging substrate 200. In one example, a piezoelectric chemical species-sensitive resonant device 250 is formed with input transducer 230, output transducer 240, base structure 236, and functionalization material 248. The input transducer 230 includes conductive vibrating structures 232 and 235 and piezoelectric material sandwiched between them. The conductive structure 232 can act as a top electrode and the region 235 of the conductive movable base structure 236 can act as a bottom electrode of the piezoelectric device. In one example, the piezoelectric material (not shown) is disposed on the bottom electrode and the top electrode is disposed on the piezoelectric material.

A piezoelectric output transducer 240 is formed with conductive structures 246 and 236 (e.g., cantilevers, beams, traces) and piezoelectric material. The conductive structure 246 can act as a second electrode and a region 237 of the conductive base structure 236 can act as a first electrode of the piezoelectric vibrating output device.

The cavity 242 can be air filled. The conductive structure 236 is anchored on one edge by package connections (e.g., anchors, vias) which may serve as both mechanical anchors as well as electrical connections to the rest of the package.

Although FIG. 2 shows one specific membrane shape, another embodiment can have other membrane shapes in order to achieve different mechanical frequencies. The membrane can also have etching holes to help with the dielectric removal process in order to create the cavity. Also, different electrode shapes can be envisioned with contacts on one or more sides of the cavity.

FIG. 3 illustrates a side view of a package substrate having a package-integrated piezoelectric chemical species-sensitive resonant device, according to an embodiment. The package substrate 300 (e.g., organic substrate) includes organic dielectric layers 302 (or layers 202) and conductive layers 321-327, 332, 336, and 346. The package substrate 300 can be formed during package substrate processing (e.g., at panel level). The package substrate 300 may represent a side view of the package substrate 200.

In one example, the package substrate 300 may be coupled or attached to multiple devices (e.g., die, chip, CPU, silicon die or chip, RF transceiver, etc.) and may also be coupled or attached to a printed circuit board (e.g., PCB 110). A cavity 342 is formed within the packaging substrate 300 by removing one or more layers (e.g., organic layers, dielectric layers, etc.) from the packaging substrate 300.

In one example, a piezoelectric transducer device 330 includes a piezoelectric stack 338 that is formed with conductive vibrating structures 332 and 336 and piezoelectric material 334. The conductive structure 332 can act as a top electrode and a region 335 of the conductive movable base structure 336 can act as a bottom electrode of the piezoelectric device. The region 335 of the base structure 336 physically contacts the piezoelectric material 334. In one example, the piezoelectric material 334 is disposed on the bottom electrode and the top electrode is disposed on the material 334. A piezoelectric output transducer 340 is formed with a stack 349 that includes conductive structures 346 and 336 (e.g., cantilevers, beams, traces) and piezoelectric material 344. The conductive structure 346 can act as a top electrode and a region 337 of the conductive base structure 336 can act as a bottom electrode of the piezoelectric output transducer.

The cavity 342 can be air filled. The conductive structure 336 is anchored on one edge by package connections 326 (e.g., anchors, vias) which may serve as both mechanical anchors as well as electrical connections to the rest of the package. The conductive structure 336 is also anchored on one edge by package connections 327 (e.g., anchors, vias) which may serve as both mechanical anchors as well as electrical connections to the rest of the package.

This structure 336 is surrounded by a cavity and is free to move in a direction (e.g., a vertical direction). In another example, the structure is free to move in a different direction. The piezoelectric film is mechanically attached to the base structure 336 and is sandwiched between the two conductive structures (electrodes). One of the electrodes can be the base structure itself.

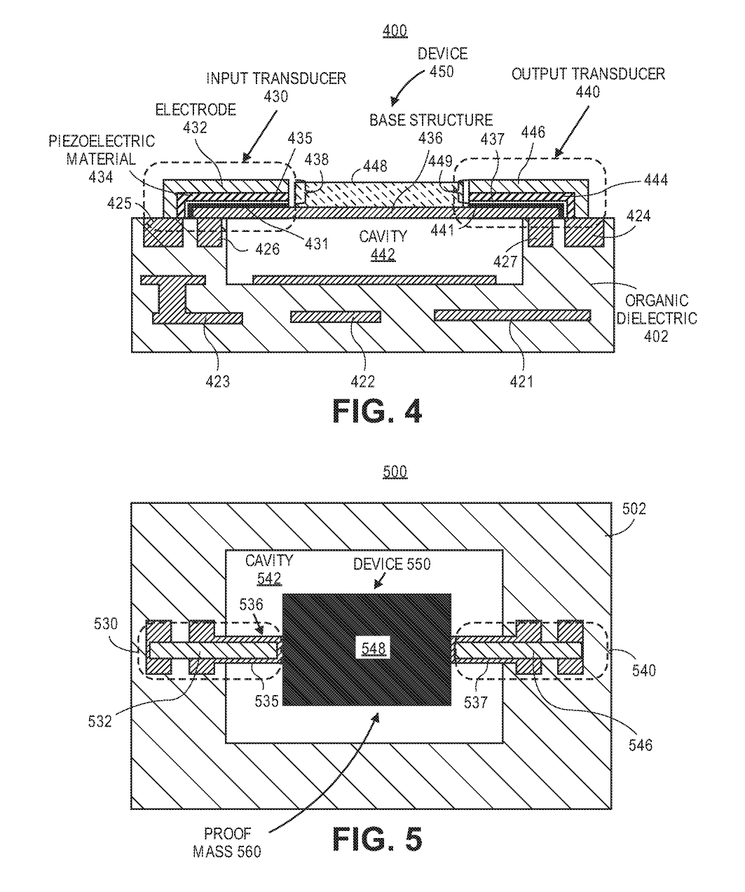

Each stack includes a piezoelectric material such as PZT, potassium sodium niobate, ZnO, or other materials sandwiched between conductive electrodes. The base structure 336 itself can be used as one of the electrodes in each stack as shown in FIG. 3, or alternatively, a separate conductive material can be used for that first electrode after depositing an insulating layer to electrically decouple this first electrode from the base structure as illustrated in FIG. 4.

FIG. 4 illustrates a side view of a package substrate having a package-integrated piezoelectric chemical species-sensitive resonant device, according to an embodiment. The package substrate 400 (e.g., organic substrate) includes organic dielectric layers 402 and conductive layers 421-427, 432, 435, 436, 437, and 446.

In one example, the package substrate 400 may be coupled or attached to multiple devices (e.g., die, chip, CPU, silicon die or chip, RF transceiver, etc.) and may also be coupled or attached to a printed circuit board (e.g., PCB 110). A cavity 442 is formed within the packaging substrate 400 by removing one or more layers (e.g., organic layers, dielectric layers, etc.) from the packaging substrate 400.

In one example, a piezoelectric transducer device 430 includes a piezoelectric stack 438 that is formed with conductive vibrating structures 432 and 435 and piezoelectric material 434. The conductive structure 432 can act as a top electrode and the conductive structure 435 can act as a bottom electrode of the piezoelectric device. The dielectric layer 431 electrically isolates the conductive structure 435 from the base structure 436. In one example, the piezoelectric material 434 is disposed on the bottom electrode and the top electrode is disposed on the material 434. A piezoelectric output transducer 440 is formed with a stack 449 that includes conductive structures 446 and 437 and piezoelectric material 444. The conductive structure 446 can act as a top electrode and the conductive structure 437 can act as a bottom electrode of the piezoelectric output transducer. The dielectric layer 441 electrically isolates the conductive structure 437 from the base structure 436.

Although piezoelectric films typically require very high crystallization temperatures that are not compatible with organic substrates, a novel process allows the deposition and crystallization of high quality piezoelectric films without heating the organic layers to temperatures that would cause their degradation. This novel process enables the integration of piezoelectric films directly in the package substrate.

For chemical sensing, a copper trace, beam, or mass (e.g., base structure 336, 436) is functionalized with a chemically selective receptor layer (e.g., functionalization material 348, 448). This layer could be any coating that preferentially adsorbs or absorbs a specific compound (or class of compounds) of interest at a higher percentage than other components in the ambient. For example, the functional layer could be a polymer coating with a high partition coefficient for a certain class of chemicals, such as volatile organic compounds (VOCs) or explosives. To increase the chemical selectivity, an array of such sensors, each with a different polymer film with different partition coefficients for different species can be used to deduce their concentrations in a mixture similar to the mechanism in a biological olfactory system. As another example, the functionalized receptor layer could be a molecular imprinted polymer (MIP), which is a polymer that is formed in the presence of a molecule that is extracted afterwards, thus leaving complementary cavities behind. These polymers show chemical affinity for the original molecule. When the template molecule is present in the environment, the template molecule attaches itself to the complementary cavity. Other molecules with different structures cannot attach to these cavities. This makes these sensors highly selective. Other examples of chemically selective receptor layers include self-assembled monolayers (SAMs) with selective terminal functional groups, antibody coatings to detect viruses, enzymes or other protein layers to detect biotoxins, and molecular sieves.

In one embodiment, a chemical species-sensitive resonant device (e.g., a chemical species sensor) operates with an input transducer being excited by applying a time varying (e.g., AC) voltage between the input electrodes of the input transducer, which causes the piezoelectric material between the electrodes to deform. The deformation induces vibrations in the base structure and these vibrations are transmitted to the output transducer. Due to the piezoelectric material in the output transducer, those transmitted vibrations generate an output electrical signal between the output electrodes.

At the output transducer, a mechanical resonant frequency of the resonant device is determined. In one method, input signals are applied to the input transducer with different frequencies and the corresponding amplitudes of the generated output signals at the output transducer are monitored. A largest amplitude of the output signal will correspond to the mechanical resonant frequency of the device. Alternatively, a feedback loop (e.g., phase locked loop) can be used to determine a mechanical resonant frequency (e.g., natural frequency) of the resonant device.

When the chemical of interest in the environment attaches itself to the functionalized material, a mass of the base structure changes and thereby changes the resonant frequency of the resonant device. The change in resonant frequency is measured using the electrical signals as described above (e.g., monitoring amplitudes of output signals, monitoring the frequency of output signals in a frequency locked loop) and used to determine the corresponding concentration or quantity of the chemical species of interest.

Alternative embodiments include using a base structure that is different from the doubly clamped straight beam of FIGS. 3 and 4 in order to attain a different range for the central frequency of the device and/or the sensitivity, or to improve the reliability of the structure. One example of alternative embodiment is shown in FIG. 5.

FIG. 5 illustrates a top view of a package substrate having a package-integrated piezoelectric chemical species-sensitive resonant device, according to an embodiment. In one example, the package substrate 500 may be coupled or attached to multiple devices (e.g., die, chip, CPU, silicon die or chip, RF transceiver, etc.) and may be also coupled or attached to a printed circuit board (e.g., PCB 110). The package substrate 500 (e.g., organic substrate) includes organic dielectric layers 502 and conductive layers 532, 536, and 546. The package substrate 500 can be formed during package substrate processing (e.g., at panel level). A cavity 542 is formed within the packaging substrate 500 by removing one or more layers (e.g., organic layers, dielectric layers, etc.) from the packaging substrate 500. In one example, a piezoelectric chemical species-sensitive resonant device 550 is formed with input transducer 530, output transducer 540, base structure 536, and a proof mass 560 having functionalization material 548 disposed on the proof mass 560. The input transducer 530 includes conductive vibrating structures 532 and 535 and piezoelectric material sandwiched between them. The conductive structure 532 can act as a top electrode and the region 535 of the conductive movable base structure 536 can act as a bottom electrode of the piezoelectric device. In one example, the piezoelectric material (not shown) is disposed on the bottom electrode and the top electrode is disposed on the piezoelectric material.

A piezoelectric output transducer 540 is formed with conductive structures 546 and 537 and piezoelectric material. The conductive structure 546 can act as a second electrode and a region 537 of the conductive base structure 536 can act as a first electrode of the piezoelectric vibrating output device.

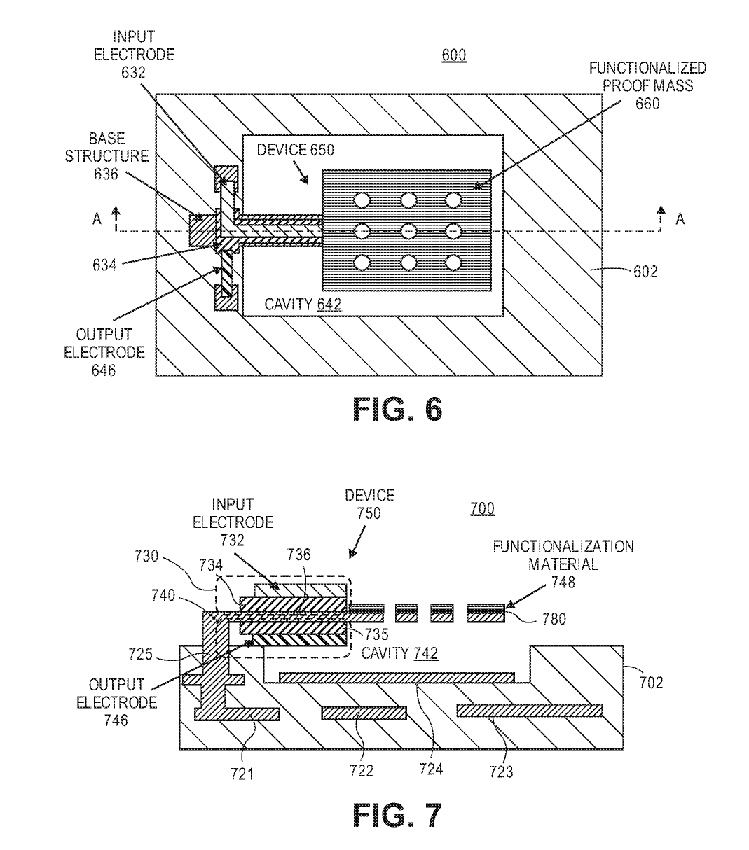

The cavity 542 can be air filled. The conductive structure 536 is anchored on one edge by package connections (e.g., anchors, vias) which may serve as both mechanical anchors as well as electrical connections to the rest of the package. This device 500 includes a base structure with a large proof mass 560 towards a center of the cavity 542. The proof mass provides additional area for applying functionalization material 548 for sensing a chemical of interest. The proof mass may have holes to facilitate etching or removal of the organic dielectric underneath to create the cavity as illustrated in FIG. 6.

FIG. 6 illustrates a top view of a package substrate having a package-integrated piezoelectric chemical species-sensitive resonant device, according to an embodiment. In one example, the package substrate 600 may be coupled or attached to multiple devices (e.g., die, chip, CPU, silicon die or chip, RF transceiver, etc.) and may be also coupled or attached to a printed circuit board (e.g., PCB 110). The package substrate 600 (e.g., organic substrate) includes organic dielectric layers 602 and conductive layers such as input electrode 632, base structure 636, and output electrode 642. The package substrate 600 can be formed during package substrate processing (e.g., at panel level). A cavity 642 is formed within the packaging substrate 600 by removing one or more layers (e.g., organic layers, dielectric layers, etc.) from the packaging substrate 600. In one example, a piezoelectric chemical species-sensitive resonant device 650 is formed with input electrode 632, piezoelectric material 634, output electrode 646, base structure 636 acting as a common electrode, and functionalized proof mass 660 having functionalization material. In one example, the proof mass 660 can have dimensions of at least 100 microns by 100 microns up to 0.5 mm by 0.5 mm.

The cavity 642 can be air filled. The conductive base structure 636 (cantilever structure) is anchored on one edge by package connections (e.g., anchors, vias) which may serve as both mechanical anchors as well as electrical connections to the rest of the package. This device 650 includes a base structure with a large proof mass 660 that provides additional area for sensing a chemical of interest.

FIG. 7 illustrates a side view (cross-sectional view AA of FIG. 6) of a package substrate having a package-integrated piezoelectric chemical species-sensitive resonant device, according to an embodiment. The package substrate 700 (e.g., organic substrate) includes organic dielectric layers 702 and conductive layers 721-725, 732, 736, and 746.

In one example, the package substrate 700 may be coupled or attached to multiple devices (e.g., die, chip, CPU, silicon die or chip, RF transceiver, etc.) and may also be coupled or attached to a printed circuit board (e.g., PCB 110). A cavity 742 is formed within the packaging substrate 700 by removing one or more layers (e.g., organic layers, dielectric layers, etc.) from the packaging substrate 700.

In one example, a piezoelectric input transducer device 730 includes a piezoelectric stack that is formed with conductive input electrode 732, base structure 736 acting as a common electrode, and piezoelectric material 734. In one example, the piezoelectric material 734 is disposed on the common electrode and the input electrode is disposed on the material 734.

A piezoelectric output transducer 740 is formed with a stack that includes base structure 736 acting as a common electrode, piezoelectric material 735, and output electrode 746.

In another embodiment, a beam of a base structure (e.g., 136, 236, 336, 436, 536, 636, 736) or a proof mass (e.g., 560, 660) for any embodiment described herein can contain a heating element (e.g., 780) that allows for the release of species from the receptor layer once a certain mass of this species has been reached on the sensor. The heater can be resistive, in the form of a conductive trace or coil which heats up when current is run through it, causing the surrounding material to heat up too. The heat can excite the species attached to the receptor layer and allow it to be released. In one example, mechanical actuation is applied simultaneously with the heat to assist in particle release. In this way, the sensor can be reused multiple times.

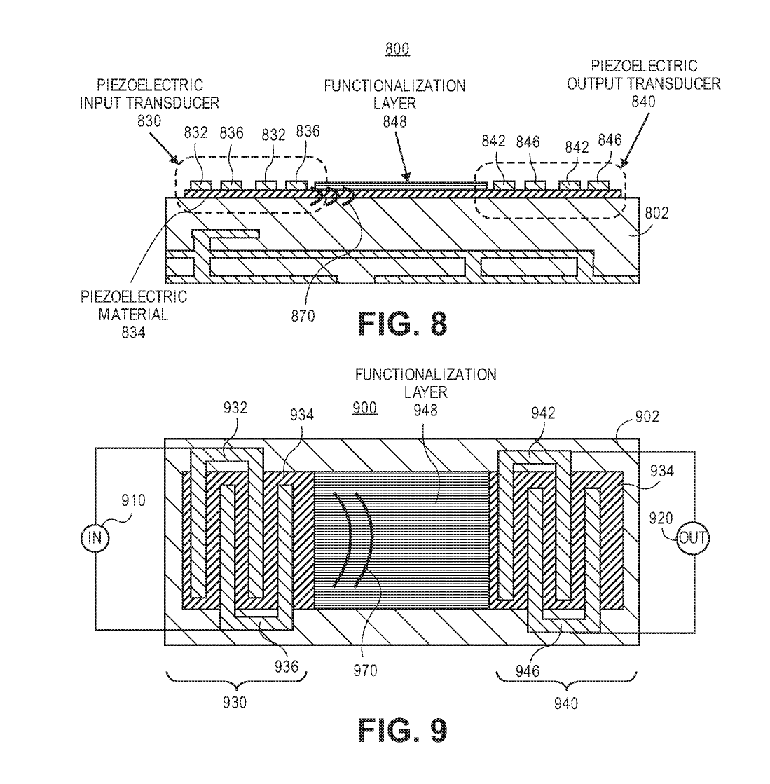

FIG. 8 illustrates a side view of a package substrate having a package-integrated piezoelectric chemical species-sensitive resonant device, according to an embodiment. The package substrate 800 (e.g., organic substrate) includes organic dielectric layers 802 and conductive layers 832, 836, 842, and 846.

In one example, the package substrate 800 may be coupled or attached to multiple devices (e.g., die, chip, CPU, silicon die or chip, RF transceiver, etc.) and may also be coupled or attached to a printed circuit board (e.g., PCB 110).

In one example, a piezoelectric chemical species-sensitive resonator is formed with input transducer 830, output transducer 840, and functionalization material 848. A piezoelectric transducer device 830 includes conductive electrodes 832 and 836 formed in a same conductive layer and piezoelectric material 834. The conductive electrode 832 can act as a first electrode and the conductive electrode 836 can act as a second electrode of the piezoelectric transducer device. Upon receiving input signals (e.g., input signal 910 of FIG. 9) which cause deformation of the piezoelectric material 834, the input transducer generates surface acoustic waves (SAW) 870 that are transmitted to the output transducer 840.

A piezoelectric output transducer 840 includes conductive electrodes 842 and 846 formed in a same conductive layer and piezoelectric material 834. The conductive electrode 842 can act as a first electrode and the conductive electrode 846 can act as a second electrode of the piezoelectric device. In response to the surface acoustic waves 870 which cause deformation of the piezoelectric material 834, the output transducer generates output signals (e.g., output signal 920 of FIG. 9).

FIG. 9 illustrates a top view of a package substrate having a package-integrated piezoelectric chemical species-sensitive resonant device, according to an embodiment. The package substrate 900 may be a top view of the package substrate 800. In one example, the package substrate 900 may be coupled or attached to multiple devices (e.g., die, chip, CPU, silicon die or chip, RF transceiver, etc.) and may be also coupled or attached to a printed circuit board (e.g., PCB 110). The package substrate 900 (e.g., organic substrate) includes organic dielectric layers 902 and conductive layers 932, 936, 942, and 946. The package substrate 900 can be formed during package substrate processing (e.g., at panel level).

In one example, a piezoelectric chemical species-sensitive resonator is formed with input transducer 930, output transducer 940, and functionalization material 948. The input transducer 930 includes conductive electrodes 932 and 936 formed in a same conductive layer and piezoelectric material 934. Upon receiving input signals which cause deformation of the piezoelectric material 934, the input transducer generates surface acoustic waves (SAW) 970 that are transmitted to the output transducer 940.

A piezoelectric output transducer 940 is formed with conductive electrodes 946 and 936 (e.g., beams, traces) formed in a same conductive layer and piezoelectric material 934. In response to the surface acoustic waves 970 which cause deformation of the piezoelectric material 934, the output transducer generates output signals.

In this embodiment, the electrodes can all be on one side of the piezoelectric material (e.g., 834, 934), such as an interdigitated transducer (IDT) configuration shown in FIGS. 8 and 9. The input transducer 930 can be used to form a surface acoustic wave (SAW) resonator with AC input signals 910, and an output IDT will measure the response with output signals 920. The chemically selective functional material 948 between the electrodes will increase in mass when the chemical of interest is adsorbed or absorbed, and therefore shift the SAW resonance frequency in a similar manner in comparison to a shift of resonant frequency for the beam-type resonators.

It will be appreciated that, in a system on a chip embodiment, the die may include a processor, memory, communications circuitry and the like. Though a single die is illustrated, there may be none, one or several dies included in the same region of the microelectronic device.

In one embodiment, the microelectronic device may be a crystalline substrate formed using a bulk silicon or a silicon-on-insulator substructure. In other implementations, the microelectronic device may be formed using alternate materials, which may or may not be combined with silicon, that include but are not limited to germanium, indium antimonide, lead telluride, indium arsenide, indium phosphide, gallium arsenide, indium gallium arsenide, gallium antimonide, or other combinations of group III-V or group IV materials. Although a few examples of materials from which the substrate may be formed are described here, any material that may serve as a foundation upon which a semiconductor device may be built falls within the scope of the present invention.

The microelectronic device may be one of a plurality of microelectronic devices formed on a larger substrate, such as, for example, a wafer. In an embodiment, the microelectronic device may be a wafer level chip scale package (WLCSP). In certain embodiments, the microelectronic device may be singulated from the wafer subsequent to packaging operations, such as, for example, the formation of one or more piezoelectric vibrating devices.

One or more contacts may be formed on a surface of the microelectronic device. The contacts may include one or more conductive layers. By way of example, the contacts may include barrier layers, organic surface protection (OSP) layers, metallic layers, or any combination thereof. The contacts may provide electrical connections to active device circuitry (not shown) within the die. Embodiments of the invention include one or more solder bumps or solder joints that are each electrically coupled to a contact. The solder bumps or solder joints may be electrically coupled to the contacts by one or more redistribution layers and conductive vias.

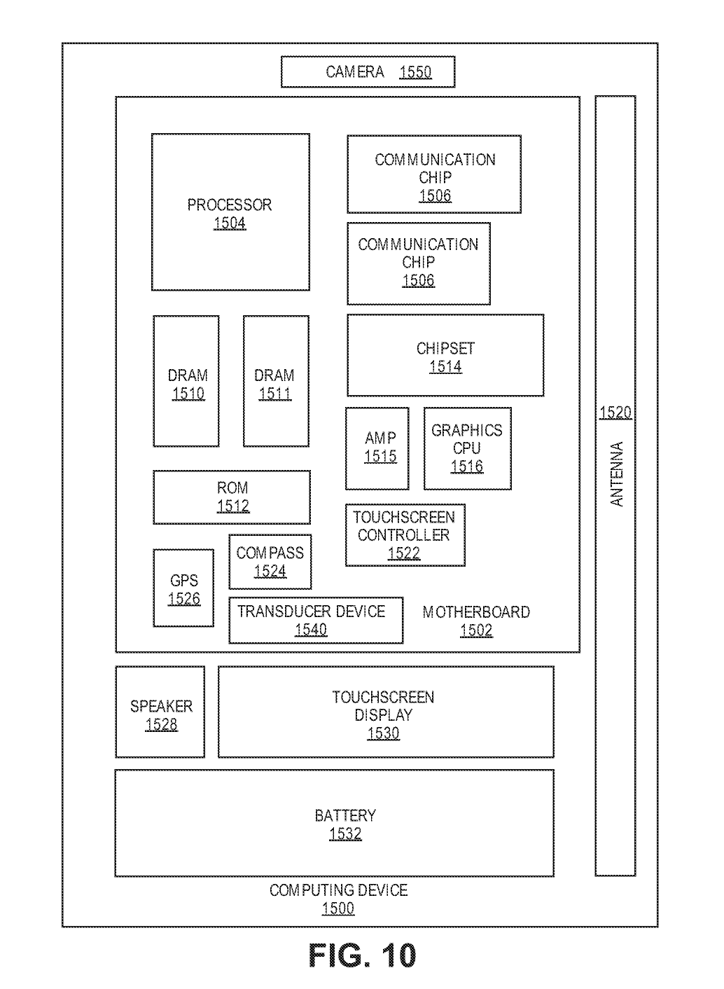

FIG. 10 illustrates a computing device 1500 in accordance with one embodiment of the invention. The computing device 1500 houses a board 1502. The board 1502 may include a number of components, including but not limited to a processor 1504 and at least one communication chip 1506. The processor 1504 is physically and electrically coupled to the board 1502. In some implementations the at least one communication chip 1506 is also physically and electrically coupled to the board 1502. In further implementations, the communication chip 1506 is part of the processor 1504.

Depending on its applications, computing device 1500 may include other components that may or may not be physically and electrically coupled to the board 1502. These other components include, but are not limited to, volatile memory (e.g., DRAM 1510, 1511), non-volatile memory (e.g., ROM 1512), flash memory, a graphics processor 1516, a digital signal processor, a crypto processor, a chipset 1514, an antenna 1520, a display, a touchscreen display 1530, a touchscreen controller 1522, a battery 1532, an audio codec, a video codec, a power amplifier 1515, a global positioning system (GPS) device 1526, a compass 1524, a chemical species-sensitive resonant device 1540 (e.g., a piezoelectric transducer device), a gyroscope, a speaker, a camera 1550, and a mass storage device (such as hard disk drive, compact disk (CD), digital versatile disk (DVD), and so forth).

The communication chip 1506 enables wireless communications for the transfer of data to and from the computing device 1500. The term “wireless” and its derivatives may be used to describe circuits, devices, systems, methods, techniques, communications channels, etc., that may communicate data through the use of modulated electromagnetic radiation through a non-solid medium. The term does not imply that the associated devices do not contain any wires, although in some embodiments they might not. The communication chip 1506 may implement any of a number of wireless standards or protocols, including but not limited to Wi-Fi (IEEE 802.11 family), WiMAX (IEEE 802.16 family), IEEE 802.20, long term evolution (LTE), Ev-DO, HSPA+, HSDPA+, HSUPA+, EDGE, GSM, GPRS, CDMA, TDMA, DECT, Bluetooth, derivatives thereof, as well as any other wireless protocols that are designated as 3G, 4G, 5G, and beyond. The computing device 1500 may include a plurality of communication chips 1506. For instance, a first communication chip 1506 may be dedicated to shorter range wireless communications such as Wi-Fi, WiGig and Bluetooth and a second communication chip 1506 may be dedicated to longer range wireless communications such as GPS, EDGE, GPRS, CDMA, WiMAX, LTE, Ev-DO, 5G, and others.

The processor 1504 of the computing device 1500 includes an integrated circuit die packaged within the processor 1504. In some implementations of the invention, the integrated circuit processor package or motherboard 1502 includes one or more devices, such as chemical species-sensitive transducer devices in accordance with implementations of embodiments of the invention. The term “processor” may refer to any device or portion of a device that processes electronic data from registers and/or memory to transform that electronic data into other electronic data that may be stored in registers and/or memory. The communication chip 1506 also includes an integrated circuit die packaged within the communication chip 1506. The following examples pertain to further embodiments.

Example 1 is a transducer device comprising a chemical species-sensitive device that includes an input transducer to receive input signals, a base structure that is coupled to the input transducer and positioned in proximity to a cavity of an organic substrate, a chemically sensitive functionalization material attached to the base structure and an output transducer to generate output signals. For a chemical sensing functionality, a desired chemical species attaches to the chemically sensitive functionalization material which causes a change in mass of the base structure and this change in mass causes a change in a mechanical resonant frequency of the device.

In example 2, the subject matter of example 1 can optionally include the chemical species-sensitive device being integrated with the organic substrate which is fabricated using panel level processing.

In example 3, the subject matter of any of examples 1-2 can optionally include the base structure being positioned above the cavity of the organic substrate to allow vibrations of the base structure.

In example 4, the subject matter of any of examples 1-3 can optionally include the output signals of the output transducer being monitored to determine the change in the mechanical resonant frequency of the device and this change in the mechanical resonant frequency correlates with an amount of the desired chemical species in an environment of the device.

In example 5, the subject matter of any of examples 1-4 can optionally include the input transducer including a first piezoelectric material which deforms upon receiving the input signals and causes the input transducer to induce vibrations in the base structure.

In example 6, the subject matter of any of examples 1-5 can optionally include the output transducer including a second piezoelectric material which deforms upon receiving the vibrations from the base structure and this causes the output signals of the output transducer to be generated.

In example 7, the subject matter of any of examples 1-6 can optionally include the mechanical resonant frequency of the device being determined by applying input signals to the input transducer with different frequencies and monitoring amplitudes of the output signals of the output transducer with a largest amplitude of the output signals corresponding to the mechanical resonant frequency.

In example 8, the subject matter of any of examples 1-7 can optionally include the base structure including a plurality of holes to increase an etch rate of organic material of the organic substrate for forming the cavity.

In example 9, the subject matter of any of examples 1-8 can optionally include the input transducer being coupled to a first electrical connection of the organic substrate in proximity to a first end of the cavity of the organic substrate and the output transducer being coupled to a second electrical connection of the organic substrate in proximity to a second end of the cavity.

In example 10, the subject matter of any of examples 1-9 can optionally include the base structure including a proof mass to increase an area for receiving the functionalization material.

Example 11 is a package substrate comprising a plurality of organic dielectric layers and a plurality of conductive layers to form the package substrate, a cavity formed in the package substrate, and a piezoelectric chemical species-sensitive device integrated within the package substrate. The piezoelectric chemical species-sensitive device including an input transducer to receive input signals, a cantilever base structure that is positioned in proximity to the cavity, a chemically sensitive functionalization material attached to the cantilever base structure and an output transducer to generate output signals. For a chemical sensing functionality, a desired chemical species attaches to the chemically sensitive functionalization material which causes a change in mass of the cantilever base structure and this change in mass causes a change in a mechanical resonant frequency of the piezoelectric chemical species-sensitive device.

In example 12, the subject matter of example 11 can optionally include the chemical species-sensitive device being integrated with the package substrate which is fabricated using panel level processing.

In example 13, the subject matter of any of examples 11-12 can optionally include the cantilever base structure being positioned above the cavity of the package substrate to allow vibrations of the cantilever base structure.

In example 14, the subject matter of any of examples 11-13 can optionally include the output signals of the output transducer being monitored to determine the change in the mechanical resonant frequency of the device and this change in the mechanical resonant frequency correlates with an amount of the desired chemical species in an environment of the device.

In example 15, the subject matter of any of examples 11-14 can optionally include the input transducer having a first piezoelectric material which deforms upon receiving the input signals and causes the input transducer to induce vibrations in the cantilever structure.

In example 16, the subject matter of any of examples 11-15 can optionally include the output transducer includes a second piezoelectric material which deforms upon receiving the vibrations from the cantilever base structure and this causes the output signals of the output transducer to be generated.

In example 17, the subject matter of any of examples 11-16 can optionally include the base structure having a plurality of holes to increase an etch rate of organic material of the organic substrate for forming the cavity.

In example 18, the subject matter of any of examples 11-17 can optionally include the cantilever base structure comprising a common electrode for the input transducer and the output transducer. The base structure includes a proof mass to increase an area for receiving the functionalization material.

Example 19 is a chemical species-sensitive resonator comprising an input transducer having a piezoelectric material to receive input signals and to generate surface acoustic waves on a surface of a package substrate having organic material in response to deformation of the piezoelectric material. A chemically sensitive functionalization material is communicatively coupled to the input transducer. An output transducer generates output signals in response to receiving the surface acoustic waves. For a chemical sensing functionality, a desired chemical species attaches to the chemically sensitive functionalization material which causes a change in mass and this change in mass causes a change in a resonant frequency of the resonator.

In example 20, the subject matter of example 19 can optionally include the chemical species-sensitive device being integrated with the package substrate which is fabricated using panel level processing.

In example 21, the subject matter of any of examples 19-20 can optionally include the output signals of the output transducer being monitored to determine the change in the resonant frequency of the resonator and this change in the resonant frequency correlates with an amount of the desired chemical species in an environment of the resonator.

Example 22 is a computing device comprising at least one processor to process data and a package substrate coupled to the at least one processor. The package substrate includes a plurality of organic dielectric layers and a plurality of conductive layers to form the package substrate which includes a piezoelectric chemical species-sensitive device having an input transducer to receive input signals, a base structure that is positioned in proximity to a cavity of the package substrate, a chemically sensitive functionalization material attached to the base structure, and an output transducer to generate output signals. For a chemical sensing functionality, a desired chemical species attaches to the chemically sensitive functionalization material which causes a change in mass of the base structure and this change in mass causes a change in a mechanical resonant frequency of the piezoelectric chemical species-sensitive device.

In example 23, the subject matter of example 22 can optionally include the chemical species-sensitive device being integrated with the organic substrate which is fabricated using panel level processing.

In example 24, the subject matter of any of examples 22-23 can optionally include a printed circuit board coupled to the package substrate.

1. A chemical species-sensitive device, comprising:
an input transducer to receive input signals;
a base structure that is coupled to the input transducer and positioned in proximity to a cavity of an organic substrate;
a chemically sensitive functionalization material attached to the base structure; and
an output transducer to generate output signals, wherein for a chemical sensing functionality a desired chemical species attaches to the chemically sensitive functionalization material which causes a change in mass of the base structure and this change in mass causes a change in a mechanical resonant frequency of the device.

2. The chemical species-sensitive device of claim 1, wherein the chemical species-sensitive device is integrated with the organic substrate which is fabricated using panel level processing.

3. The chemical species-sensitive device of claim 2, wherein the base structure is positioned above the cavity of the organic substrate to allow vibrations of the base structure.

4. The chemical species-sensitive device of claim 1, wherein the output signals of the output transducer are monitored to determine the change in the mechanical resonant frequency of the device and this change in the mechanical resonant frequency correlates with an amount of the desired chemical species in an environment of the device.

5. The chemical species-sensitive device of claim 1, wherein the input transducer includes a first piezoelectric material which deforms upon receiving the input signals and causes the input transducer to induce vibrations in the base structure.

6. The chemical species-sensitive device of claim 5, wherein the output transducer includes a second piezoelectric material which deforms upon receiving the vibrations from the base structure and this causes the output signals of the output transducer to be generated.

7. The chemical species-sensitive device of claim 1, wherein the mechanical resonant frequency of the device is determined by applying input signals to the input transducer with different frequencies and monitoring amplitudes of the output signals of the output transducer with a largest amplitude of the output signals corresponding to the mechanical resonant frequency.

8. The chemical species-sensitive device of claim 1, wherein the base structure includes a plurality of holes to increase an etch rate of organic material of the organic substrate for forming the cavity.

9. The chemical species-sensitive device of claim 1, wherein the input transducer is coupled to a first electrical connection of the organic substrate in proximity to a first end of the cavity of the organic substrate and the output transducer is coupled to a second electrical connection of the organic substrate in proximity to a second end of the cavity.

10. The chemical species-sensitive device of claim 7, wherein the base structure includes a proof mass to increase an area for receiving the functionalization material.

11. A package substrate comprising:
a plurality of organic dielectric layers and a plurality of conductive layers to form the package substrate;
a cavity formed in the package substrate; and
a piezoelectric chemical species-sensitive device integrated within the package substrate, the piezoelectric chemical species-sensitive device including an input transducer to receive input signals, a cantilever base structure that is positioned in proximity to the cavity, a chemically sensitive functionalization material attached to the cantilever base structure and an output transducer to generate output signals, wherein for a chemical sensing functionality a desired chemical species attaches to the chemically sensitive functionalization material which causes a change in mass of the cantilever base structure and this change in mass causes a change in a mechanical resonant frequency of the piezoelectric chemical species-sensitive device.

12. The package substrate of claim 11, wherein the chemical species-sensitive device is integrated with the package substrate which is fabricated using panel level processing.

13. The package substrate of claim 12, wherein the cantilever base structure is positioned above the cavity of the package substrate to allow vibrations of the cantilever base structure.

14. The package substrate of claim 11, wherein the output signals of the output transducer are monitored to determine the change in the mechanical resonant frequency of the device and this change in the mechanical resonant frequency correlates with an amount of the desired chemical species in an environment of the device.

15. The package substrate of claim 11, wherein the input transducer includes a first piezoelectric material which deforms upon receiving the input signals and causes the input transducer to induce vibrations in the cantilever structure.

16. The package substrate of claim 15, wherein the output transducer includes a second piezoelectric material which deforms upon receiving the vibrations from the cantilever base structure and this causes the output signals of the output transducer to be generated.

17. The package substrate of claim 11 wherein the base structure includes a plurality of holes to increase an etch rate of organic material of the organic substrate for forming the cavity.

18. The package substrate of claim 11, wherein the cantilever base structure comprises a common electrode for the input transducer and the output transducer, wherein the base structure includes a proof mass to increase an area for receiving the functionalization material.

19. A chemical species-sensitive resonator, comprising:
an input transducer having a piezoelectric material to receive input signals and to generate surface acoustic waves on a surface of a package substrate having organic material in response to deformation of the piezoelectric material;
a chemically sensitive functionalization material communicatively coupled to the input transducer; and
an output transducer to generate output signals in response to receiving the surface acoustic waves, wherein for a chemical sensing functionality a desired chemical species attaches to the chemically sensitive functionalization material which causes a change in mass and this change in mass causes a change in a resonant frequency of the resonator.

20. The chemical species-sensitive resonator of claim 19, wherein the chemical species-sensitive device is integrated with the package substrate which is fabricated using panel level processing.

21. The chemical species-sensitive resonator of claim 19, wherein the output signals of the output transducer are monitored to determine the change in the resonant frequency of the resonator and this change in the resonant frequency correlates with an amount of the desired chemical species in an environment of the resonator.

22. A computing device comprising:
at least one processor to process data; and
a package substrate coupled to the at least one processor, the package substrate includes a plurality of organic dielectric layers and a plurality of conductive layers to form the package substrate which includes a piezoelectric chemical species-sensitive device having an input transducer to receive input signals, a base structure that is positioned in proximity to a cavity of the package substrate, a chemically sensitive functionalization material attached to the base structure, and an output transducer to generate output signals, wherein for a chemical sensing functionality a desired chemical species attaches to the chemically sensitive functionalization material which causes a change in mass of the base structure and this change in mass causes a change in a mechanical resonant frequency of the piezoelectric chemical species-sensitive device.

23. The computing device of claim 22, wherein the chemical species-sensitive device is integrated with the organic substrate which is fabricated using panel level processing.

24. The computing device of claim 22, further comprising:
a printed circuit board coupled to the package substrate.
